(19) United States
(12) Patent Application Publication
(10) Pub. No.: US 2024/0416096 A1
(43) Pub. Date: Dec. 19, 2024

(54) Systems and Methods for Angled Insertion of Microneedles for Targeted Delivery

(71) Applicant: Georgia Tech Research Corporation, Atlanta, GA (US)

(72) Inventors: Mark R. Prausnitz, Atlanta, GA (US); Isabella Bowland, Boulder, CO (US); Rohan Murty, Dublin, CA (US); Abishek Sankaranarayanan, Atlanta, GA (US)

(21) Appl. No.: 18/700,816

(22) PCT Filed: Oct. 14, 2022
(86) PCT No.: PCT/US2022/046796

§ 371 Date: Apr. 12, 2024

Related U.S. Application Data
(60) Provisional application No. 63/255,853, filed on Oct. 14, 2021.

(51) Int. Cl. A61M 37/00; A61B 17/20

(52) U.S. Cl. CPC A61M 37/0015; A61B 17/205; A61M 2037/0023; A61M 2037/0046; A61M 2037/0053; A61M 2037/0061; A61M 2202/07; A61M 2202/30; A61M 2205/0238

(57) ABSTRACT

A method for targeting epidermal delivery of an agent of interest is provided, where the method includes inserting at least one microneedle into the skin of a patient at a non-perpendicular angle, the at least one microneedle having the agent of interest. The at least one microneedle may be inserted into the skin of a patient using a system including the at least one microneedle and an angled insertion apparatus.

### CROSS-REFERENCE TO RELATED APPLICATIONS

This application claims priority to U.S. Provisional Patent Application No. 63/255,853, filed Oct. 14, 2021, which is incorporated herein by reference.

### BACKGROUND

Administration of pharmaceuticals, cosmeceuticals, and other agents into the skin can be advantageous for a number of different outcomes. Specifically, targeted delivery to the epidermis of the skin (i.e., the viable epidermis) is appealing when a site of action of the agent being delivered is located in the epidermis. The epidermis is also an appealing site for targeted delivery because the epidermis does not have blood vessels, which means that systemic uptake and distribution of the agent is reduced.

Some agents are delivered to the skin to treat conditions in the skin or to achieve cosmetic outcomes. In some of those cases, the site of action for the agent is in the epidermis, and systemic delivery of the agent can cause side effects, waste of drug or other undesirable outcomes. Other agents may be designed to generate responses in the body that are not for treating the skin, but those responses can be initiated in the skin. For example, an antigen can be delivered to the skin to generate an immune response that has systemic effects. The antigen may be taken up by cells of the immune system in the skin as part of the initiation of the immune response.

While targeted delivery to epidermis is desirable, it is hard to do. The epidermis of human skin is 50 to 100 microns thick, which is thinner than the width of most hypodermic needles, making intraepidermal injection extremely difficult. An agent can be placed on the surface of the skin, where it can cross the stratum corneum and thereby access the viable epidermis. However, very few agents have suitable properties to cross stratum corneum at useful rates. There are various methods known in the art to increase delivery across the stratum corneum (e.g., chemical enhancers, iontophoresis, ultrasound, liposomes) but they all have drawbacks, including low efficiency of delivery and requirement for delivery over long periods of time.

Conventional methods for treating peanut allergies include subcutaneous allergen immunotherapy (SCIT) and oral immunotherapy (OIT). These immunotherapies are based on extended exposure of the immune system to low doses of antigen to induce immunological tolerance to the antigen, thereby suppressing allergic responses. SCIT is often cumbersome, as subcutaneous injection of antigens, such as Arachis hypogaea 2—the dominant peanut allergen—can cause severe adverse effects in patients. Adverse effects stemming from SCIT are caused by systemic uptake into the bloodstream and rapid antigen clearance by macrophages in the dermis and subcutaneous space. These processes increase the production of IgE antibodies, causing mast cells to flood the system with histamines; in extreme cases, this may result in anaphylaxis. In contrast, delivery to the avascular epidermis reduces systemic uptake, thereby increasing the relative production of IgG₄ antibodies—i.e., those responsible for immune tolerance.

Additionally, some people, particularly children, may suffer from needle phobia. While OIT avoids injections, it is still in relatively early development and is hindered by patients' increased risk of anaphylaxis and gastrointestinal discomfort, leading to withdrawal from treatment and, thus, resulting in the rapid reversal of allergen desensitization. In recent years, topical immunotherapies that avoid the issues of needle phobia and gastrointestinal adverse effects have emerged (e.g., Viaskin by DBV Technologies, Bagneux, France), but they generally suffer from poor efficacy and regulatory barriers.

It would be desirable to improve both the safety and efficacy of targeted allergen immunotherapy within the viable epidermis, as this would significantly decrease the rate of systemic uptake and elicit the desired immune response. It also would be desirable to provide a method of administering these and other agents, e.g., those for dermatological therapies, preferentially to the epidermis in a rapid and efficient manner, preferably while reducing needle phobia concerns.

### BRIEF SUMMARY

In one aspect, a method is provided for targeting epidermal delivery of an agent of interest. The method may include inserting one or more microneedles into the skin of a patient, wherein the one or more microneedles include an antigen, a drug, or another agent of interest, and wherein the one or more microneedles are inserted at an angle that is non-perpendicular to the skin surface. In particular embodiments, angled insertion of the microneedle into the skin enables more superficial delivery that targets antigen deposition in the epidermis compared to perpendicular microneedle insertion, such that the present methods and systems may be effective to localize at least 50%, and sometimes at least 70%, 80% or 90%, of the antigen in the epidermis.

In another aspect, a system is provided which includes at least one microneedle and an insertion apparatus configured to guide the at least one microneedle into the skin of a patient at a non-perpendicular angle. The at least one microneedle may include an antigen or other agent of interest and the at least one microneedle may be configured to release the antigen or other agent of interest into the skin following insertion into the skin. In some embodiments, the insertion apparatus includes a first member having a planar surface that is configured to rest against a skin surface, and a guide member having an insertion end from which the at least one microneedle extends, wherein the guide member is configured to translate relative to the first member to insert the at least one microneedle into the skin surface at a selected non-perpendicular angle.

In another aspect, a method for allergen immunotherapy is provided. The method may include: inserting an array of microneedles comprising an antigen into the skin of a patient, wherein the microneedle has a length and is in inserted at a non-perpendicular angle effective to locate a majority of the inserted length of the microneedles within the epidermis of the skin; and permitting the antigen to be released from the inserted microneedle into the epidermis.

In another aspect, a method for skin treatment is provided, for dermatological, cosmetic or other purposes. The method may include: inserting an array of microneedles comprising a drug or cosmeceutical agent into the skin of a person, wherein the microneedle has a length and is in inserted at a non-perpendicular angle effective to locate a majority of the inserted length of the microneedles within the epidermis of the skin; and permitting the drug or cosmeceutical agent to be released from the inserted microneedle into the epidermis.

### BRIEF DESCRIPTION OF THE DRAWINGS

The detailed description is set forth with reference to the accompanying drawings. The use of the same reference numerals may indicate similar or identical items. Various embodiments may utilize elements and/or components other than those illustrated in the drawings, and some elements and/or components may not be present in various embodiments. Elements and/or components are not necessarily drawn to scale.

FIG. 1 illustrates embodiments of conventional perpendicular insertion of a hypodermic needle and a microneedle as compared to the angled microneedle insertion of the present disclosure.

FIG. 2 is cross-sectional view of a linear array of coated microneedles, according to one embodiment of the present disclosure.

FIG. 3A is a perspective view of a system that includes a microneedle array and an insertion apparatus, according to an embodiment of the present disclosure.

FIGS. 3B and 3C are front view of the system shown in FIG. 3A, with the guide member and microneedles in two different positions to illustrate the displacement that occurs during insertion of the microneedles, according to an embodiment of the present disclosure.

FIGS. 4A and 4B are side views of a system that includes a microneedle array and an insertion apparatus placed onto a skin surface, which is adjustable to select different angles of insertion of the microneedles into the skin, according to an embodiment of the present disclosure.

### DETAILED DESCRIPTION

Methods and systems have been developed for targeting epidermal delivery of essentially any agent of interest. The method includes inserting one or more microneedle into the skin of a patient, wherein the one or more microneedles include an agent of interest (for example as part of a dissolvable coating on the microneedles), wherein the one or more microneedles are inserted at angle that is non-perpendicular to the skin surface, preferably wherein a majority (i.e., greater than 50%) of the inserted length of the at least one microneedle is located within the viable epidermis of the skin. For example, 60%, 70%, 80%, or 90%, or more of the microneedle length is within the viable epidermis following the insertion of the microneedle which has a length that is, for example, more than twice the thickness of the epidermis.

As used herein, the terms “epidermis” and “epidermal” refer to the viable epidermis, not including the stratum corneum, whether or not the term “viable” is explicitly recited.

Peanut and tree nut allergies account for most food-induced anaphylactic events. The standard allergy immunotherapy approach involves subcutaneous injection, which is challenging because severe adverse reactions can occur when antigens spread systemically. Allergen localization within the epidermis (i.e., the upper 20-100 μm of skin) should significantly reduce systemic uptake, because the epidermis is avascular. Microneedles can be adapted to provide an effective approach of targeting antigen delivery to the epidermis.

However, while a conventional microneedle patch provides a convenient method for drug delivery to the skin, they generally target both the epidermis and the dermis, leading to systemic delivery. Microneedle patches have been shown to alleviate many of the translational barriers associated with SCIT, as their size/geometry provides an attractive opportunity for targeting antigen delivery to epidermal thicknesses (˜100 μm) and administration in the form of a microneedle patch can eliminate or reduce the issue of needle phobia. However, conventional microneedle patches for drug delivery involve delivery to both the epidermis and dermis, which is undesirable for allergy immunotherapy. In addition, it may be difficult to limit microneedle penetration depth by using microneedles that are shorter than epidermis thickness, at least because (i) antigen loading on such small microneedles may be too low to deliver the required dose and (ii) controlled insertion of such short microneedles is difficult to achieve without a specialized high-velocity insertion device. Conventionally, microneedles are inserted perpendicular to the surface of the skin. This often leads to localization in the dermis (100-2000 μm from surface) and is not preferred for allergy immunotherapy applications.

The epidermis is not only an attractive site for immune responses but is also a site where many dermatological problems exist and can be treated. In topical dermatological drugs, a challenge is always getting high concentrations in the skin while minimizing systemic exposures. Accordingly, in some embodiments using the presently disclosed methods, a dermatological drug (where the site of action is in the epidermis) is delivered to the epidermis for a dermatological therapy and targeted to deliver the drug primarily or exclusively to the viable epidermis, in order to avoid or minimize an administered drug getting into systemic circulation, which may lead to negative side effects.

Similarly, in other embodiments of the present method, the agent of interest delivered to the viable epidermis is a vaccine or cosmeceutical agent with a site of action in the epidermis.

It has been discovered that angled insertion of, for example, 250 μm microneedles may be effective to achieve a shallower insertion depth. This angled insertion may be superior to simply inserting shorter microneedles perpendicularly since it affords greater loading of antigen (or another agent of interest) (i.e., because the longer angled microneedles have more surface area and volume than the shorter microneedles needed for perpendicular insertion) and improves repeatability of insertions to target the epidermis (0-100 μm from surface), which induces the desired immune (or other desired biological) response while mitigating the risk of adverse reactions.

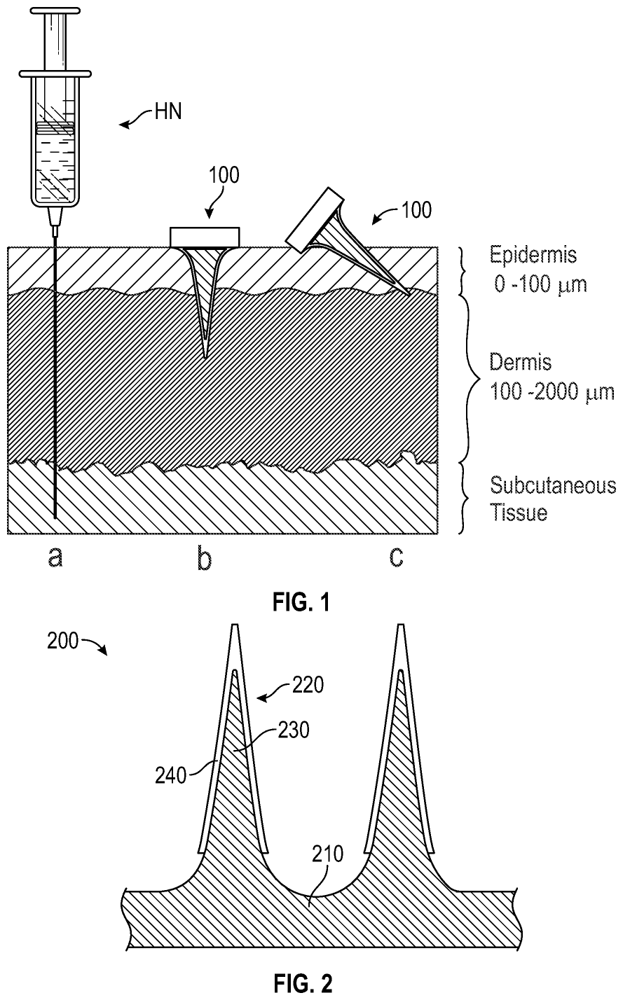

FIG. 1 illustrates how the two conventional approaches of perpendicular insertion of a hypodermic needle (HN) and a microneedle 100 (on the left side and middle of the figure) penetrate well into the dermis, whereas with an angled insertion of a microneedle 100 (on the right side of the figure), most of the microneedle remains in epidermis.

With the present disclosure, improved microneedles and insertion methods are adapted for epidermal localization. In some embodiments, this is accomplished by performing angled insertion of the microneedles that limits the microneedle insertion entirely or mostly to the epidermis and provides a microneedle length, e.g., 200 μm, 250 μm, or more, that provides a microneedle size that is sufficiently large for carrying an effective dose of agent for delivery and can be inserted using a simple procedure, which may be a manual procedure.

In some embodiments of the present disclosure, a method is provided that includes inserting one or more microneedle into the skin of a patient, wherein the one or more microneedles include an agent of interest (for example as part of a dissolvable coating on the microneedles) and have a length that is more than 150% (e.g., from 200% to 400%) of the thickness of the epidermis, wherein the one or more microneedles are inserted in a manner that the inserted microneedles, and/or the agent of interest, are localized within the epidermis.

In some embodiments of the method, the inserted microneedles generally remain inserted in the skin (e.g., in the epidermis) for a period effective to release the agent of interest therein. The period may be long enough to dissolve a coating off of a microneedle or long enough to permit the microneedle to dissolve or separate from a base structure. In some embodiments, this period may be from a few seconds to a few minutes, for example from 30 seconds to 5 minutes. Dissolution of coating on a microneedle may be faster (e.g., 30 seconds to 90 seconds) than dissolution of a dissolvable microneedle.

Many skin treatments could benefit from epidermal targeting. For example, dermatological disorders, such as basal and squamous cell carcinomas, cutaneous warts, and vitiligo, have pathology in the epidermis which means that epidermal delivery can increase drug efficacy. Furthermore, because the viable epidermis (possibly mediated by the basal membrane at the dermal-epidermal junction) provides a significant barrier to diffusion of macromolecules, protein delivered to epidermis may be retained there. While other molecules may more readily cross the dermal-epidermal junction, targeted delivery to the avascular epidermis may still limit delivery to dermis and the vasculature located there.

In a particular embodiment, the method is used in allergy immunotherapy, for which targeted epidermal delivery is expected to facilitate generating immune tolerance while reducing the risk of adverse side effects from systemic antigen exposure. In addition, such a microneedle-based allergy immunotherapy may reduce the need for SCIT, democratizing allergy treatment in several ways. For example, immunotherapy using a microneedle array may substantially lower the treatment cost, may mitigate the issue of needle phobia, which may also increase patient compliance and expand the patient base for non-oral food-allergy immunotherapy, and if the microneedle array for immunotherapy can be administered at home, parents may eliminate many trips to the doctor by administering the allergen themselves.

### Agents of Interest

The present methods and insertion systems can be used to deliver essentially any suitable substance, or agent of interest, for epidermal targeting and/or allergy immunotherapy. As used herein, the term “agent of interest” includes active pharmaceutical ingredients, allergens, vitamins, cosmetic agents, cosmeceuticals, diagnostic agents, markers (e.g., colored dyes or radiological dyes or markers), and other materials that are desirable to introduce into a biological tissue. The “agent of interest” may be referred to as a drug or an active agent. The agent of interest may be a prophylactic, therapeutic, or diagnostic agent useful in medical or veterinary application. The agent of interest may be a prophylactic or therapeutic substance, which may be referred to herein as an API. In some embodiments, the API is selected from suitable proteins, peptides and fragments thereof, which can be naturally occurring, synthesized or recombinantly produced. In some embodiments, the agent of interest includes a vaccine. Examples of vaccines include vaccines for infectious diseases, therapeutic vaccines for cancers, neurological disorders, allergies, and smoking cessation or other addictions. The API may be selected from small molecules and larger biotechnology produced or purified molecules (e.g., peptides, proteins, DNA, RNA).

In some embodiments, the agent of interest is an allergen useful in treating a peanut or tree nut allergy. For example, the allergen may be or include Ara h 2, the protein responsible for most peanut-induced reactions.

### Microneedles

Microneedles useful in the present methods for administering an agent of interest can be made using a variety of methods known in the art, and the agent of interest can be operably associated with the microneedle by various means known in the art. For example, the agent of interest can be included as part of a dissolvable coating composition on the surface of the microneedle, or the agent of interest can be part of a dissolvable composition forming the microneedle structure itself.

The delivery method may use a single microneedle or an array of two or more microneedles. The microneedles may be solid or hollow. The array may be a linear (co-planar) array of two to twenty microneedles. The array may contain exactly two, three, four, five, six, seven, eight, nine, ten, eleven, or twelve microneedles, for example.

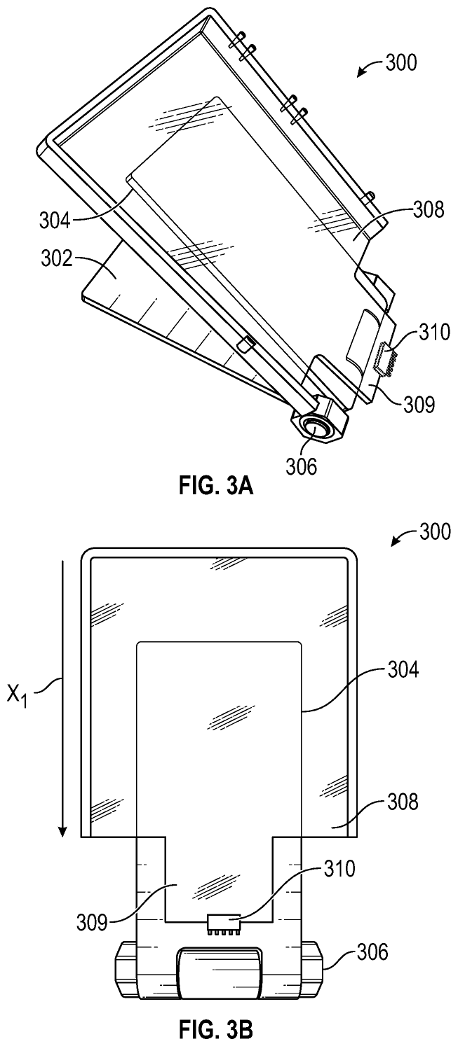

FIG. 2 illustrates one embodiment of a linear array of microneedles having a coating composition that comprises an agent of interest. The coating may cover all or part of the exterior surface of each microneedle. For example, the coating may be only on the upper part (i.e., the part distal to the base) of each microneedle, for instance covering about 75% of the microneedle length.

Coated microneedles may be convenient for the application of inducing allergic tolerance since the coating solution may be modified to include whichever antigen the allergy immunotherapy is treating. For example, the microneedles may be coated with Ara h 2, the protein responsible for most peanut-induced reactions, to treat such reactions. In some embodiments of a method for inducing allergic tolerance to peanuts, the microneedles are configured to deliver a dose of 50 μg of antigen. The dose may also be greater or less than this amount in other embodiments. Accordingly, the number of microneedles needed, or the number of individual microneedle insertions needed, may depend on the amount of antigen able to be loaded onto/into, and released from, each microneedle.

The microneedles may be made of essentially any suitable biocompatible material, which may be a metal, polymeric, or ceramic material. In some embodiments, the microneedles are constructed by using wet-etching photo-lithographically defined needle structures from stainless-steel sheets, and then the resulting stainless steel microneedles are coated with a coating solution containing an agent of interest, which is subsequently dried to yield a microneedle having a solid coating layer containing the agent of interest. In preferred embodiments, the coating layer is readily dissolvable in vivo, e.g., in the dermis.

In some preferred embodiments, the microneedles have a length from 200 to 300 microns, such as 250 microns, in order to achieve an insertion where most of the microneedle is located in the epidermis upon substantially complete insertion whereby most or all of agent of interest is released in the epidermis. Other lengths of microneedles may be suitable, however, for use in the angled insertion methods described herein.

### Insertion Systems

The present methods can be carried out by any suitable means for directing and inserting the at least one microneedle into the skin at a non-perpendicular angle. In preferred embodiments, a system, or apparatus, is provided to consistently and accurately guide the microneedle into the skin at a selected non-perpendicular angle. The apparatus may include mechanical adjustment feature to adjust the insertion angle to two or more different angles, or the apparatus may be fixed to provide only a single predetermined insertion angle.

In some embodiments, the system includes at least one microneedle which comprises an antigen or other agent of interest; and an insertion apparatus configured to guide the at least one microneedle into the skin of a patient at a non-perpendicular angle, wherein the at least one microneedle is configured to release the antigen or other agent of interest into the skin following insertion into the skin. In some embodiments, the insertion apparatus includes (i) a first member having a surface (e.g., planar or curved, for example in a shape to conform to patient's arm) that is configured to rest against a skin surface; and (ii) a guide member having an insertion end from which the at least one microneedle extends, wherein the guide member is configured to translate relative to the first member to insert the at least one microneedle into the skin surface at a selected non-perpendicular angle. The guide member may cooperate with the first member in essentially any manner that holds the position of the microneedle at the selected insertion angle as the microneedle travels in a direction along the longitudinal axis of the microneedle toward and into the skin. In some embodiments, the insertion apparatus further includes a second member, which may be planar, connected to the first member, and the guide member is configured to translate along a surface of the second member. The surface of the second member may be planar. In some embodiments, the first member and the second member are connected at an end of each member by a hinge which is configured to permit the second member to rotate and adjust the selected non-perpendicular angle at which the at least one microneedle is inserted.

In some embodiments, the one or more microneedles are removably attached to the guide member of the insertion apparatus. In this way, the microneedles can be replaced after use, and the insertion apparatus can be re-used for multiple microneedle insertions.

In some embodiments, the microneedles of the system include a base from which the microneedles extend and a handling tab which can be used to attach/detach the microneedles to the guide member of the insertion apparatus. In a preferred embodiment, the system includes a linear array of two or more microneedles, such as from four to 20 microneedles. In a preferred embodiment, these microneedles each have a length from 200 to 300 microns, such as 250 microns.

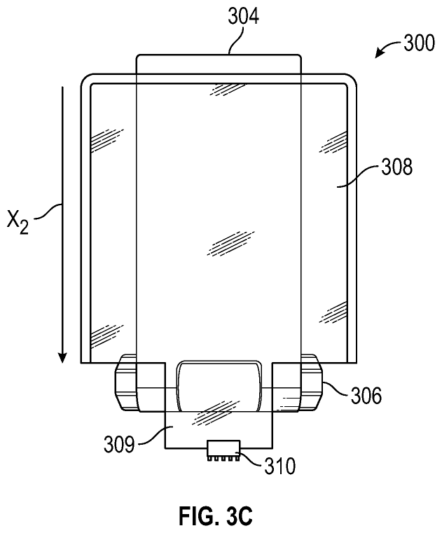

FIGS. 3A-3C illustrate one embodiment of an adjustable microneedle insertion system 300. The system 300 includes a first member 302 having a planar surface that is configured to rest against a skin surface, a second member 304 connected to the first member 302 at hinge 306. The system 300 further includes a guide member 308 that has an insertion end 309 from which a co-planar array of microneedles 310 extends. The guide member 308 is configured to translate relative to the first and second members along a surface of the second member 304 to drive the insertion end 309 toward a skin surface to insert the microneedles 310 into the skin surface at a selected angle, e.g., between 90° and 5°. In the illustrated embodiment, the hinge 306 includes a includes a bolt and nut which can be loosened to permit the second member to rotate with respect to the first member and thereby change the angle defined between the first and second members. Once at the desired angle, the nut can be tightened to secure the positions of the first and second members relative to one another.

FIGS. 3B and 3C show the system 300 with the guide member 308 and microneedle array 310 at a first, retracted position (X₁) (FIG. 3B) and with the guide member 308 and microneedle array 310 at a second, extended position (X₂) (FIG. 3C). The arrow shows the direction of translation between the two positions for microneedle insertion.

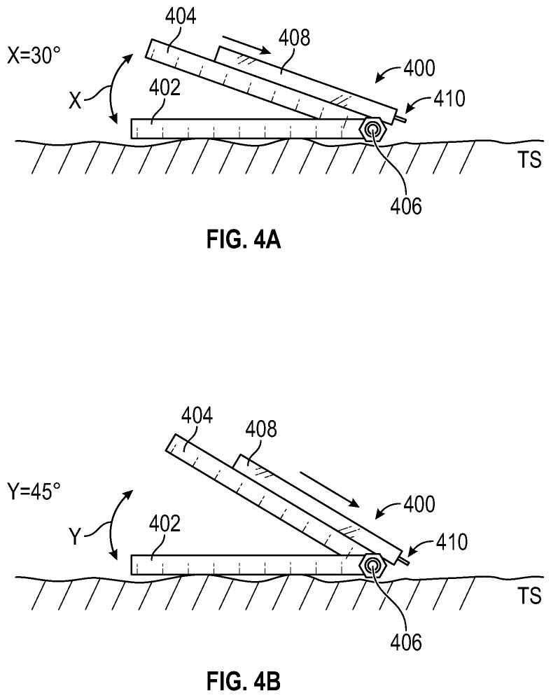

The adjustability of the insertion angle is illustrated in FIGS. 4A and 4B, which show an insertion system 400 at two different angular positions. The insertion system 400 includes a first member 402 having a planar surface that is resting against a skin surface TS. The system 400 further includes a second member 404 connected to the first member 402 at hinge 406 and a guide member 408 from which microneedle 410 extends. The guide member 404 is configured to translate in the direction shown by the arrow along a surface of the second member 404 to drive the microneedle 410 toward and into a skin surface, at a 30° angle in FIG. 4A or at 45° angle in FIG. 4B. The guide member can translate in the opposite direction to remove the microneedle from the skin (or any remaining portion thereof, e.g., a base, in cases where the microneedle dissolves in vivo).

The invention can be further understood with reference to following non-limiting examples.

### Example 1: Making Antigen Coated Microneedles

Solid stainless-steel microneedle arrays were constructed by using wet-etching photo-lithographically defined needle structures from stainless-steel sheets (Tech Etch, Plymouth, MA or Moonlight Therapeutics, Atlanta, GA, USA). Each microneedle array consisted of five co-planar MNs, each measuring 250 μm in length. Prior to coating, the microneedles were treated with oxygen plasma (Plasma Cleaner PDC-32G, Harrick Plasma, Ithaca, NY, USA) to increase the steel surface's hydrophilicity and improve coating-solution adhesion. An aqueous coating solution was prepared, which consisted of 2% (w/v) medium-viscosity carboxymethylcellulose sodium salt (Sigma-Aldrich, St. Louis, MO, USA) and 0.5% (w/v) Pluronic F-68 (Sigma-Aldrich). The coating solution also contained 2% (w/v) peanut Arachis hypogaea (Greer Laboratories, Lenoir, NC, USA) and/or 0.2% (w/v) sulforhodamine B sodium salt (Sigma-Aldrich) as a red-fluorescent dye. Specifically, the coating solution was prepared by adding 20 mg medium viscosity CMC and 5 mg Pluronic F-68, as well as 10 mg peanut protein and/or 2 mg sulforhodamine B dye to 1 mL deionized (DI) water, and then this solution was mixed via vortexing and sonication.

The stainless steel microneedle arrays were mounted onto a custom-made automated dip-coating device (see Gill, H.S.; Prausnitz, M.R. Coated microneedles for transdermal delivery. J. Control. Release 2007, 117, 227-37.) with which the microneedles were dip-coated in the coating solution, using two-axis robotic control. Each microneedle in the array was coated 10 times, following a procedure described in Li, S.; Li, W.; Prausnitz, M. Individually coated microneedles for co-delivery of multiple compounds with different properties. Drug Deliv. Transl. Res. 2018, 8, 1043-52. The microneedle arrays were air-dried and stored at ambient conditions (20-25° C., 30-60% relative humidity) prior to insertion. The amount of antigen coated onto each microneedle was determined by dissolving the microneedle coating in DI water and measuring antigen concentration by bicinchoninic acid assay (see Thermo Fisher Scientific. Pierce BCA Protein Assay Kit User Guide 2020. Available online: https://assets.thermofisher.com/TFS-Assets/LSG/manuals/MAN0011430_Pierce_BCA_Protein_Asy_UG.pdf (accessed on 26 December 2021).

### Example 2: Angled Insertion for Epidermal Targeting

A shallow insertion depth with increased epidermal targeting was achieved by using a 250 μm long microneedle inserted at an acute angle to the skin surface (20°) that enabled an insertion depth less than 100 μm and epidermal targeting of up to 70%.

Insertion of Microneedles into Porcine Skin Ex Vivo

Porcine belly skin was obtained from Pel-Freez Biologicals, (Rogers, AR, USA), and the subcutaneous fat layer was removed. The skin surface was dabbed with Kim wipes to remove excess moisture, and then fixed tautly onto a cutting board to simulate natural skin tension during microneedle insertion. The microneedles were pressed into the skin for 30 seconds and then removed. Perpendicular insertions (i.e., insertions at a 90° angle) were performed by hand. Angled insertions at 45° and 20° were executed by using the adjustable insertion apparatus described in FIGS. 3A-3C.

### Histological Analysis

Immediately after insertion, the tissue around the microneedle array insertion site was excised and prepared for histological sectioning by freezing in liquid nitrogen for 1-2 minutes in Optimal Cutting Temperature Embedding Medium for Frozen Tissue Specimens (Scigen Scientific, Gardena, CA, USA). The angle of insertion was also marked on the outside of the block for guidance during sectioning before being stored at −20° C. until sectioning. Because small-molecule dye diffusion happens on the timescale of minutes (and the large protein diffuses much slower), significant diffusion of the antigen away from the MN insertion site was not expected.

Within minutes (or hours at most), histological analysis was conducted to assess microneedle insertion depth into the skin and image distribution of materials dissolved from the microneedle coatings in the skin. Samples were sectioned to 10-15 μm thickness by using a Leica 3050 S Cryostat (Wetzlar, Germany), with 3-5 sections being collected for each of the five insertion sites imprinted by each microneedle array.

Frozen sections were imaged by using a stereo microscope (Olympus SZX16, Tokyo, Japan) with red fluorescence to visualize and quantify the depth of delivery of sulforhodamine. The Autostainer XL (Leica) was used to stain sections with hematoxylin and eosin (H&E), which were then imaged by using the same microscope under bright field to determine the extent of epidermal localization.

### Optical Coherence Tomography

Microneedles inserted into skin were imaged in real time by using OCT (VivoSight Dx, Michelson Diagnostics, Kent, United Kingdom) with infrared light (1305 nm) to visualize to skin depths up to 500 μm from the skin surface. The OCT scan was configured to produce 500 slices of a 6 mm×6 mm section of the skin.

### Statistical Analysis

Statistical analysis was performed by using OriginPro 2021b software (Northampton, MA, USA). One-way ANOVA tests were used to evaluate statistical differences between the groups for insertion depth, percent epidermal targeting, and percent peanut antigen localization in the epidermis. Significance was considered for p<0.05.

### Coating Microneedles With Peanut Protein Antigen

A formulation and method for coating a suitable dose of peanut antigen on microneedle arrays was developed. Four factors were varied to meet a target antigen loading of 0.5 μg per microneedle: concentration of peanut protein and CMC in the coating solution, microneedle length, and number of dip-coating cycles.

The aqueous coating solution consisted of the peanut protein, CMC, and Pluronic F-68. While the Pluronic concentration was kept constant at 0.5% (w/v), the antigen concentration was varied from 1% to 3% (w/v), and the CMC concentration from 1% to 2% (w/v). Increasing protein concentration from 1% to 2% nearly doubled the amount of antigen loading (to ˜0.6 μg/microneedle), but higher concentrations did not significantly improve the loading. Protein concentrations above ˜2% were not pursued because they formed a highly viscous, hard-to-mix coating solution that yielded unreliable coating results. For CMC, a 2% (w/v) concentration afforded a greater antigen loading compared to 1% (w/v), probably because the increased coating solution viscosity kept more coating solution on the microneedle surface during drying.

Varying microneedle length from 200 to 450 μm caused a 250% increase in total antigen coating. Because longer microneedles could be coated with more antigen and 200 μm long microneedles met the loading target of 0.5 μg/microneedle, 250 μm long microneedles would also achieve sufficient loading, as well as expected skin insertion.

Increasing the number of dip-coating cycles from 5 to 15 also caused a >200% increase in antigen loading. However, adding more dip-coating cycles increased the likelihood of antigen coating below the base of the microneedle. This is not desirable, because any coating not on the microneedles themselves will not enter the skin upon insertion. As such, 10 coating cycles resulted in sufficient antigen loading, while mitigating the frequency of coating past the base.

The optimized coating method employed a formulation containing 2% (w/v) CMC, 0.5% (w/v) Pluronic F-68, 1% (w/v) peanut antigen, and 0.2% (w/v) sulforhodamine B (to assist coating visualization) that was coated onto 250 μm long stainless-steel microneedles, using 10 dip-coating cycles. Because the target dose was achieved with 1% peanut antigen, that concentration was used instead of higher concentrations; however, as long as changes to the formulation do not hinder the solution's ability to coat uniformly on microneedle tips (e.g., by becoming too viscous), varying either the CMC or protein amount should not affect epidermal targeting. Although 250 μm microneedles were not specifically studied in these coating experiments, 200 μm microneedles were long enough to achieve the target loading, so any microneedle equal or greater in length would be sufficient from a dosing perspective. When considering insertion, however, 250 μm microneedles were superior, because reliable insertion of 200 μm (or shorter) microneedles was difficult to achieve. Thus, the final microneedle array was selected to be long enough to achieve the target dose and insert reliably at shallow angles, while being short enough not to penetrate too far into the dermis.

### Microneedle Insertion Depth Into Skin After Angled Insertion

It was hypothesized that angled insertion of microneedle s into the skin enables more superficial delivery that targets antigen deposition in the epidermis compared to perpendicular microneedle insertion.

To limit microneedle insertion depth primarily to the epidermis (which is approximately 20-100 μm thick in humans), two strategies were combined. First, short microneedles of 250 μm length were used. Second, the microneedles were inserted at an angle of 90°,45°, or 20°. By geometry, these microneedles are expected to insert 250 μm, 177 μm, and 85 μm into the skin (i.e., insertion depth (D) equals microneedle length (L) times the sin of the insertion angle (θ), D=L sin θ).

Histological analysis of microneedle insertions showed that microneedle insertion depth decreased with decreasing insertion angle (one-way ANOVA, p<0.001). As expected for perpendicular insertion, the mean depth was 265±45 μm, which is not significantly different from the microneedle length of 250 μm (within 95% confidence interval of 230-300 μm). After perpendicular insertion, deformation of the skin surface was evident, with the microneedle insertion site located at the base of a valley on the skin surface, which is consistent with prior reports (see, e.g., Martanto, W.; Moore, J.S.; Couse, T.; Prausnitz, M.R. Mechanism of fluid infusion during microneedle insertion and retraction. J. Control. Release 2006, 112, 357-361).

The decrease in insertion depth caused by an insertion angle of 45° yielded an average depth of 207±20 μm. Although this is consistent with the expectation that angled insertions reduced insertion depth, this insertion depth was not preferred for epidermal targeting. Moreover, the reduction in insertion depth was not as great as that predicted by the simple geometric calculation of 177 μm (outside the 95% confidence interval of 184-217 μm). It is not clear why this calculated value was not predictive, but it probably has to do with the non-rigid nature of skin tissue that does not conform to the shape of a perfect right triangle.

Further decreasing the insertion angle to 20° lowered insertion depth to 97±15 μm, which is in the range of human epidermal thickness (˜100 μm), thus suggesting a viable way to increase the extent of epidermal targeting with microneedles. In addition, this insertion depth is not significantly different from the calculated depth of 85 μm (within 95% confidence interval of 83-112 μm).

The degree of variability of insertion depth and found that the coefficient of variation was ˜10% after 90° and 45° insertion and was ˜15% after 20° insertion was also considered. Sources of variability may be due to microneedles sometimes “jumping” across the tissue or sometimes bending due to imperfect rigidity of the MNs, causing less uniform delivery. It was noted that these effects were more prevalent during insertion at very acute angles, and this may explain the greater variability after 20° insertion. Other sources of variability include irregularities in the skin, pushing microneedles too hard during insertion, performing perpendicular insertions by hand, and imperfect estimation of insertion depths from histological images. Nonetheless, variation was relatively small and suggests the ability to achieve targeted depth of insertion with good reliability.

### Efficiency of Epidermal Targeting by Angled Microneedle Insertion

Although controlling microneedle insertion depth was a primary focus, assessing the degree of epidermal targeting was also assessed. The skin was stained with H&E to identify the epidermis as a dark purple layer, while staining the dermis a pink/salmon color. In this way, the degree of epidermal localization was determined as the insertion site's interfacial contact length with the purple-stained regions divided by the total perimeter of the insertion site within the tissue. While direct measurement of epidermal delivery would be ideal, this necessitates separating the stratum corneum, epidermis, and dermis layers through interventions, such as incubating skin with enzymes or heating skin to 50-60° C. This could introduce artefacts, such as increased dye diffusion, likely inefficient dye/protein extraction efficiency, and mechanical disruption of the tissue. Consequently, an imaging-based method that provides a reasonable estimate of the degree of epidermal targeting was selected instead.

Decreasing the insertion angle significantly increased epidermal targeting (one-way ANOVA, p<0.001). Perpendicular insertion only afforded 25%±13% localization in the epidermis, whereas reducing the insertion angle to 45° improved this localization nearly twofold (42%±13%). Acute angle insertion at 20° further increased epidermal targeting to 70%±21%.

The coefficient of variation was highest for the perpendicularly inserted microneedles; this can be explained by the natural morphology of the porcine epidermis, which is structurally similar to the human epidermis. The epidermis has an undulating finger-like structure, with epidermal thickness varying as a function of position. When microneedles were inserted perpendicularly to the skin surface, they randomly localized either in one of the finger-like protrusions of the epidermis or in one of the “troughs”, causing significant variability in the interfacial contact distance at each insertion site. In contrast, microneedles inserted at an angle almost always pierced through several of these finger-like protrusions, thereby decreasing the variability. Additionally, microneedles were sometimes bent during insertion, and this served to increase epidermal localization because the direction in which the microneedles were bent resulted in shallower insertion depths. Because the force was always applied from above, the microneedles were consistently bent in a predictable manner, tending to be parallel to the skin's surface, thus leading to shallower depths of insertion.

### OCT Imaging of Angled Microneedle Insertion Into Skin

To complement that analysis performed on histological skin sections stained after microneedle insertion, the skin was also imaged by OCT to visualize microneedles embedded in situ in the skin. In these images, the microneedles were seen as a bright line extending from the upper right corner. While the skin is present across the whole bottom half of the images, it could only be seen on the left side, because the right side was shadowed from view by the angled insertion device positioned between the skin and the OCT imaging head. In these images, the microneedle can be seen penetrating into the skin at an angle reaching depths of approximately 100 μm after 20° insertion and approximately 200 μm after 45° insertion, which is in general agreement with the data from histological sections.

### Conclusion

Angled microneedle insertion can be used to target antigen delivery to the upper layers of the skin, notably the epidermis. The microneedle coating method was first developed to achieve a target dose of 0.5 μg peanut protein antigen (Arachis hypogaea) per microneedle by adjusting coating formulation composition, microneedle length, and number of dip-coating cycles. Using a custom 3D-printed biplanar angular insertion device, microneedles were inserted into porcine skin ex vivo with controlled insertion angles of 20°, 45°, and 90°. Histological analysis determined that angled insertion at 20° reduced mean depth of microneedle penetration into skin from 265 to 97 μm compared to perpendicular) (90° insertion; this result was consistent with additional imaging performed by OCT. The extent of epidermal targeting increased from 25% after perpendicular delivery to 70% after delivery at a 20° angle. Thus, angled microneedle insertion provides a promising approach to targeting drug delivery to the epidermis that may improve allergy immunotherapy and enable other applications with epidermal delivery targets.

Some embodiments of the present disclosure can be described in view of one or more of the following:
Embodiment 1 includes a method for targeting epidermal delivery of an agent of interest, the method comprising: inserting at least one microneedle into the skin of a patient, the at least one microneedle comprising the agent of interest, wherein the at least one microneedle is inserted at an angle that is non-perpendicular to the skin surface.
Embodiment 2 may include Embodiment 1, wherein a majority of the inserted length of the at least one microneedle is located within the epidermis of the skin.
Embodiment 3 may include Embodiment 1 or 2, wherein the agent of interest is an antigen.
Embodiment 4 may include any one of Embodiments 1 to 3, wherein the antigen is one selected for allergy immunotherapy.
Embodiment 5 may include any one of Embodiments 1 to 4, wherein the antigen comprises a protein associated with peanut-induced allergic reactions.
Embodiment 6 may include any one of Embodiments 1 to 4, wherein the agent of interest is a vaccine or a drug which has biological activity in the viable epidermis for a dermatological treatment.
Embodiment 7 may include any one of Embodiments 1 to 6, wherein the agent of interest is at least part of a coating on the exterior of the at least one microneedle.
Embodiment 8 may include any one of Embodiments 1 to 7, wherein the agent of interest is dispersed within a dissolvable matrix material forming the at least one microneedle.
Embodiment 9 may include any one of Embodiments 1 to 8, wherein the at least one microneedle has a length from 200 to 300 microns, such as 250 microns.
Embodiment 10 may include any one of Embodiments 1 to 9, wherein the at least one microneedle is a linear array of a plurality of microneedles.
Embodiment 11 may include any one of Embodiments 1 to 10, wherein the at least one microneedle is inserted at an angle between 5 degrees and 90 degrees, such as between 5 degrees and 45 degrees.
Embodiment 12 may include any one of Embodiments 1 to 10, wherein the at least one microneedle is inserted at an angle from 15 degrees to 45 degrees or from 10 degrees to 30 degrees, such as from 20 degrees to 30 degrees.
Embodiment 13 may include any one of Embodiments 1 to 12, wherein: the at least one microneedle comprises an antigen or a drug, and the at least one microneedle is inserted at an angle that is non-perpendicular to the skin surface and effective to localize at least 50% of the antigen or drug in the epidermis.
Embodiment 14 includes a system, or apparatus, configured to carry out any of the methods of Embodiments 1 to 13.
Embodiment 15 includes a system comprising: at least one microneedle which comprises an antigen or other agent of interest; and an insertion apparatus configured to guide the at least one microneedle into the skin of a patient at a non-perpendicular angle, wherein the at least one microneedle is configured to release the antigen or other agent of interest into the skin following insertion into the skin.
Embodiment 16 may include the system of Embodiment 15, wherein the insertion apparatus comprises: a first member having a planar surface that is configured to rest against a skin surface; a guide member having an insertion end from which the at least one microneedle extends, wherein the guide member is configured to translate relative to the first member to insert the at least one microneedle into the skin surface at a selected non-perpendicular angle.
Embodiment 17 may include Embodiment 16, wherein the insertion apparatus further comprises a second member connected to the first member, and the guide member is configured to translate along a surface of the second member.
Embodiment 18 may include Embodiment 17, wherein the first member and the second member are connected at end of each member by a hinge which is configured to permit the second member to rotate and adjust the selected non-perpendicular angle at which the at least one microneedle is inserted.
Embodiment 19 may include any one of Embodiments 14 to 18, wherein the at least one microneedle is a linear array of two or more microneedles, such as from four to 20 microneedles.
Embodiment 20 may include any one of Embodiments 14 to 19, wherein the at least one microneedle has a length from 200 to 300 microns, such as 250 microns.
Embodiment 21 may include any one of Embodiments 16 to 20, wherein the at least one microneedle is removably attached to the guide member.
Embodiment 22 may include any one of Embodiments 14 to 21, wherein: the at least one microneedle comprises an antigen, which may be part of a coating on the at least one microneedle, and the angle of insertion and the at least one microneedle is configured to localize at least 50% of the antigen in the epidermis of the skin.
Embodiment 23 may include any one of Embodiments 14 to 22, wherein the antigen comprises a protein associated with peanut-induced allergic reactions.
Embodiment 24 is a method for allergen immunotherapy, wherein the method comprises: inserting an array of microneedles comprising an antigen into the skin of a patient, wherein the microneedle has a length and is in inserted at a non-perpendicular angle effective to locate a majority of the inserted length of the microneedles within the viable epidermis of the skin; and releasing the antigen from the inserted microneedle into the viable epidermis.
Embodiment 25 is a method for dermatological treatment, wherein the method comprises: inserting an array of microneedles comprising a drug, which has biological activity in the viable epidermis, into the skin of a patient, wherein the microneedle has a length and is in inserted at a non-perpendicular angle effective to locate a majority of the inserted length of the microneedles within the viable epidermis of the skin; and releasing the drug from the inserted microneedle into the viable epidermis.
Embodiment 26 may include Embodiment 24 or 25, wherein a majority of the inserted length of the at least one microneedle is located within the epidermis of the skin.
Embodiment 27 may include any one of Embodiments 24 to 26, wherein the agent of interest is an antigen, for example wherein the antigen comprises a protein associated with peanut-induced allergic reactions.
Embodiment 28 may include any one of Embodiments 24 to 26, wherein the agent of interest is a vaccine or a drug which has biological activity in the viable epidermis for a dermatological treatment.
Embodiment 29 may include any one of Embodiments 24 to 28, wherein the agent of interest is at least part of a coating on the exterior of the at least one microneedle.
Embodiment 30 may include any one of Embodiments 24 to 29, wherein the agent of interest is dispersed within a dissolvable matrix material forming the at least one microneedle.
Embodiment 31 may include any one of Embodiments 24 to 30, wherein the at least one microneedle has a length from 200 to 300 microns, such as 250 microns.
Embodiment 32 may include any one of Embodiments 24 to 31, wherein the at least one microneedle is a linear array of a plurality of microneedles.
Embodiment 33 may include any one of Embodiments 24 to 32, wherein the at least one microneedle is inserted at an angle between 5 degrees and 90 degrees, such as between 5 degrees and 45 degrees.
Embodiment 34 may include any one of Embodiments 24 to 33, wherein the at least one microneedle is inserted at an angle from 15 degrees to 45 degrees or from 10 degrees to 30 degrees, such as from 20 degrees to 30 degrees.
Embodiment 35 may include Embodiment 15, wherein the insertion apparatus is designed to insert the at least one microneedle into the skin surface at only a single selected non-perpendicular angle.

Modifications and variations of the methods and devices described herein will be obvious to those skilled in the art from the foregoing detailed description. Such modifications and variations are intended to come within the scope of the appended claims.

1. A method for targeting epidermal delivery of an agent of interest, the method comprising:
inserting at least one microneedle into the skin of a patient, the at least one microneedle comprising the agent of interest,
wherein the at least one microneedle is inserted at an angle that is non-perpendicular to the skin surface.

2. The method of claim 1, wherein a majority of the inserted length of the at least one microneedle is located within the viable epidermis of the skin.

3. The method of claim 1, wherein the agent of interest is an antigen.

4. The method of claim 3, wherein the antigen is one selected for allergy immunotherapy.

5. The method of claim 4, wherein the antigen comprises a protein associated with peanut-induced allergic reactions.

6. The method of claim 1, wherein the agent of interest is a vaccine or a drug which has biological activity in the viable epidermis for a dermatological treatment.

7. The method of claim 1, wherein the agent of interest is part of a coating on the exterior of the at least one microneedle.

8. The method of claim 1, wherein the agent of interest is dispersed within a dissolvable matrix material forming the at least one microneedle.

9. The method of claim 1, wherein the at least one microneedle has a length from 200 to 300 microns.

10. The method of claim 1, wherein the at least one microneedle is a linear array of a plurality of microneedles.

11. The method of claim 10, wherein the at least one microneedle is inserted at an angle between 5 degrees and 45 degrees.

12. The method of claim 11, wherein the at least one microneedle is inserted at an angle from 10 degrees to 30 degrees.

13. The method of claim 1, wherein:
the at least one microneedle comprises an antigen, and
the at least one microneedle is inserted at an angle that is non-perpendicular to the skin surface and effective to localize at least 50% of the antigen in the viable epidermis.

14. A system comprising:
at least one microneedle which comprises an antigen, drug or other agent of interest; and
an insertion apparatus configured to guide the at least one microneedle into the skin of a patient at a non-perpendicular angle,
wherein the at least one microneedle is configured to release the antigen, drug or other agent of interest into the skin following insertion into the skin.

15. The system of claim 14, wherein the insertion apparatus comprises:
a first member having a surface that is configured to rest against a skin surface; and
a guide member having an insertion end from which the at least one microneedle extends,
wherein the guide member is configured to translate relative to the first member to insert the at least one microneedle into the skin surface at a selected non-perpendicular angle.

16. The system of claim 15, wherein the insertion apparatus further comprises a second member connected to the first member, and the guide member is configured to translate along a surface of the second member.

17. The system of claim 16, wherein the first member and the second member are connected at an end of each member by a hinge which is configured to permit the second member to rotate and adjust the selected non-perpendicular angle at which the at least one microneedle is inserted.

18. The system of claim 14, wherein the at least one microneedle is a linear array of two or more microneedles.

19. The system of claim 14, wherein the at least one microneedle has a length from 200 to 300 microns.

20. The system of claim 14, wherein the at least one microneedle is removably attached to the guide member.

21. The system of claim 14, wherein the insertion apparatus is designed to insert the at least one microneedle into the skin surface at only a single selected non-perpendicular angle.

22. The system of claim 14, wherein:
the at least one microneedle comprises a coating comprising an antigen or a drug, and
the angle of insertion and the at least one microneedle are configured to localize at least 50% of the antigen or drug in the viable epidermis of the skin.

23. The system of claim 22, wherein the antigen comprises a protein associated with peanut-induced allergic reactions.

24. The system of claim 22, wherein the drug has biological activity in the viable epidermis for a dermatological treatment.

25. The system of claim 14, wherein the insertion apparatus is configured to guide the at least one microneedle into the skin of a patient at an angle between 5 degrees and 45 degrees.

26. (canceled)

27. A method for allergen immunotherapy, the method comprising:
inserting an array of microneedles comprising an antigen into the skin of a patient, wherein the microneedle has a length and is in inserted at a non-perpendicular angle effective to locate a majority of the inserted length of the microneedles within the viable epidermis of the skin; and
releasing the antigen from the inserted microneedle into the viable epidermis.

28. A method for dermatological treatment, the method comprising:
inserting an array of microneedles comprising a drug, which has biological activity in the viable epidermis, into the skin of a patient, wherein the microneedle has a length and is in inserted at a non-perpendicular angle effective to locate a majority of the inserted length of the microneedles within the viable epidermis of the skin; and
releasing the drug from the inserted microneedle into the viable epidermis.
